(19) United States
(12) Patent Application Publication
(10) Pub. No.: US 2021/0290336 A1
(43) Pub. Date: Sep. 23, 2021

(54) METHOD AND SYSTEM FOR PERFORMING SURGICAL IMAGING BASED ON MIXED REALITY

(71) Applicant: TAIWAN MAIN ORTHOPAEDIC BIOTECHNOLOGY CO., LTD., Taichung City (TW)

(72) Inventor: Min-Liang WANG, Taichung City (TW)

(21) Appl. No.: 17/205,382

(22) Filed: Mar. 18, 2021

(30) Foreign Application Priority Data
Mar. 20, 2020 (TW) 109109496

(51) Int. Cl. A61B 90/00; A61B 90/50; G06T 19/00

(52) U.S. Cl. CPC A61B 90/361; A61B 90/39; A61B 90/50; G06T 19/006; G06T 2219/2016; A61B 2090/367; A61B 2090/3979; A61B 2090/502; A61B 2090/365

(57) ABSTRACT

A method for performing surgical imaging based on mixed reality (MR) includes: obtaining a 3D virtual model of a body part of a subject, the 3D virtual model including a plurality of model reference points; continuously capturing IR images of the body part, including a plurality of IR reference points; calculating a first projection matrix based on the IR images; continuously capturing color images of the body part; calculating a second projection matrix based on the color images; in response to a calibration operation, calculating a third projection matrix; and generating a to-be-projected model using the 3D virtual model and the projection matrices.

### CROSS-REFERENCE TO RELATED APPLICATION

This application claims priority of Taiwanese Patent Application No. 109109496, filed on Mar. 20, 2020.

### FIELD

The disclosure relates to a method and a system for performing surgical imaging based on mixed reality.

### BACKGROUND

Conventionally, in a surgical procedure, a particular area of a subject (e.g., a patient) that requires surgery may not be directly visible to the surgeon performing the surgery. In such cases, surgical imaging procedures such as magnetic resonance imaging (MRI), or computed tomography (CT) may be implemented to create a three-dimensional (3D) virtual model of the particular area, which can be displayed on a screen to assist the surgeon. However, such configuration of displaying the 3D virtual model requires frequent head turns by the surgeon to look at the screen so as to make sure the surgical instruments (e.g., a scalpel, a drill, a tube, etc.) are accurately placed.

A more convenient setup for the surgeon to perform the surgery uses a mixed reality (MR) system to present the 3D virtual model directly on an MR device (e.g., a headset with a display screen, which is to be worn by the surgeon) such that to the perception of the surgeon, the 3D virtual model is superimposed on the body of the subject.

### SUMMARY

One object of the disclosure is to provide a method for performing surgical imaging based on mixed reality (MR) for assisting a user in performing a surgical procedure.

According to the disclosure, the method for performing surgical imaging based on mixed reality (MR) is implemented using a system that includes an MR device to be worn by a user. The MR device includes a processor, an infrared (IR) image capturing unit, a color image capturing unit and a display lens. The method includes:

a) obtaining, by the processor, a three-dimensional (3D) virtual model of a body part of a subject, the 3D virtual model including a plurality of model reference points that are associated with a plurality of marks on the body part, respectively;

b) controlling, by the processor, the IR image capturing unit to continuously capture IR images of the body part of the subject, each of the IR images of the body part including a plurality of IR reference points that correspond in location with the marks on the body part, respectively;

c) calculating, by the processor, a first projection matrix based on a plurality of mark coordinate sets associated respectively with the plurality of marks on the body part in a global 3D coordinate system, and a plurality of IR coordinate sets associated respectively with the plurality of IR reference points in a first two-dimensional (2D) coordinate system;

d) controlling, by the processor, the color image capturing unit to continuously capture color images of the body part of the subject, each of the color images of the body part including a plurality of color reference points that correspond in location with the marks on the body part, respectively;

e) obtaining, by the processor, a plurality of color coordinate sets associated respectively with the plurality of color reference points in a second 2D coordinate system;

f) calculating, by the processor, a second projection matrix based on the plurality of mark coordinate sets and the plurality of color coordinate sets;

g) controlling, by the processor, the display lens to display a plurality of calibration points, and an instruction for instructing the user to perform a calibration operation with respect to each of the calibration points, to thereby obtain a plurality of screen coordinate sets that are associated respectively with the plurality of calibration points on the display lens and a plurality of calibrated coordinate sets that are associated with the calibration points in the global 3D coordinate system;

h) calculating, by the processor, a third projection matrix based on the plurality of screen coordinate sets and the plurality of calibrated coordinate sets;

i) generating, by the processor, a to-be-projected model by performing a projection operation on the 3D virtual model, the projection operation being performed based on a plurality of original pixel coordinate sets, the first projection matrix, the second projection matrix and the third projection matrix, the plurality of original pixel coordinate sets being associated respectively with a plurality of pixels that constitute the 3D virtual model in the global 3D coordinate system; and

j) controlling, by the processor, the display lens to display the to-be-projected model.

Another object of the disclosure is to provide a mixed reality (MR) system that is capable of implementing the above-mentioned method.

According to the disclosure, the MR system for performing surgical imaging includes an electronic device and an MR device that communicates with said electronic device and that is to be worn by a user. The electronic device includes a processor, a data storage, a communication unit, an input interface and a display screen. The MR device includes a processor, an infrared (IR) image capturing unit, a color image capturing unit and a display lens.

The processor of the electronic device is programmed to obtain a three-dimensional (3D) virtual model of a body part of a subject, the 3D virtual model including a plurality of model reference points that are associated with a plurality of marks on the body part, respectively.

The processor of the MR device is programmed to control the IR image capturing unit to continuously capture IR images of the body part of the subject. Each of the IR images of the body part includes a plurality of IR reference points that correspond in location with the marks on the body part, respectively.

The processor of the electronic device is programmed to obtain a first projection matrix based on a plurality of mark coordinate sets associated respectively with the plurality of marks on the body part in a global 3D coordinate system, and a plurality of IR coordinate sets associated respectively with the plurality of IR reference points in a first two-dimensional (2D) coordinate system.

The processor of the MR device is programmed to control the color image capturing unit to continuously capture color images of the body part of the subject. Each of the color images of the body part includes a plurality of color reference points that correspond in location with the marks on the body part, respectively.

The processor of the electronic device is programmed to obtain a plurality of color coordinate sets associated respectively with the plurality of color reference points in a second 2D coordinate system, and to calculate a second projection matrix based on the plurality of mark coordinate sets and the plurality of color coordinate sets.

The processor of the MR device is programmed to control the display lens to display a plurality of calibration points, and an instruction for instructing the user to perform a calibration operation with respect to each of the calibration points, to thereby obtain a plurality of screen coordinate sets that are associated respectively with the plurality of calibration points on the display lens and a plurality of calibrated coordinate sets that are associated with the calibration points in the global 3D coordinate system.

The processor of the electronic device is programmed to calculate a third projection matrix based on the plurality of screen coordinate sets and the plurality of calibrated coordinate sets, and to generate a to-be-projected model by performing a projection operation on the 3D virtual model. The projection operation is performed based on a plurality of original pixel coordinate sets, the first projection matrix, the second projection matrix and the third projection matrix, the plurality of original pixel coordinate sets being associated respectively with a plurality of pixels that constitute the 3D virtual model in the global 3D coordinate system.

The processor of the MR device is programmed to control the display lens to display the to-be-projected model.

Another object of the disclosure is to provide a non-transitory computer-readable storage medium storing instructions that, when executed by a processor of an electronic device communicating with a mixed reality device, cause the processor to perform steps of the aforementioned method.

### BRIEF DESCRIPTION OF THE DRAWINGS

Other features and advantages of the disclosure will become apparent in the following detailed description of the embodiments with reference to the accompanying drawings, of which:

FIG. 1 is a schematic diagram of a system for performing surgical imaging based on mixed reality according to one embodiment of the disclosure;

FIG. 2 is a block diagram illustrating components of the system according to one embodiment of the disclosure;

FIG. 3 is a flowchart illustrating steps of a method for performing surgical imaging based on mixed reality according to one embodiment of the disclosure;

FIG. 4 is a flowchart illustrating sub-steps of an operation for obtaining a plurality of original pixel coordinate sets from a three-dimensional virtual model; and

FIG. 5 is a flowchart illustrating sub-steps of an operation for generating a to-be-projected model by performing a projection operation on the three-dimensional virtual model.

### DETAILED DESCRIPTION

Before the disclosure is described in greater detail, it should be noted that where considered appropriate, reference numerals or terminal portions of reference numerals have been repeated among the figures to indicate corresponding or analogous elements, which may optionally have similar characteristics.

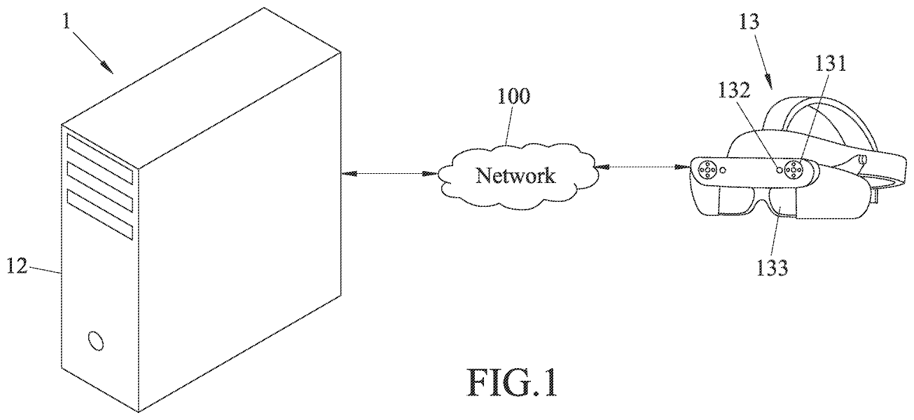

FIG. 1 is a schematic view of a system 1 for performing surgical imaging based on mixed reality according to one embodiment of the disclosure. In this embodiment, the system 1 includes an electronic device 12 and a mixed reality (MR) device 13.

The electronic device 12 may be embodied using a personal computer (PC), a server computer, a laptop, a tablet, a smartphone, etc.

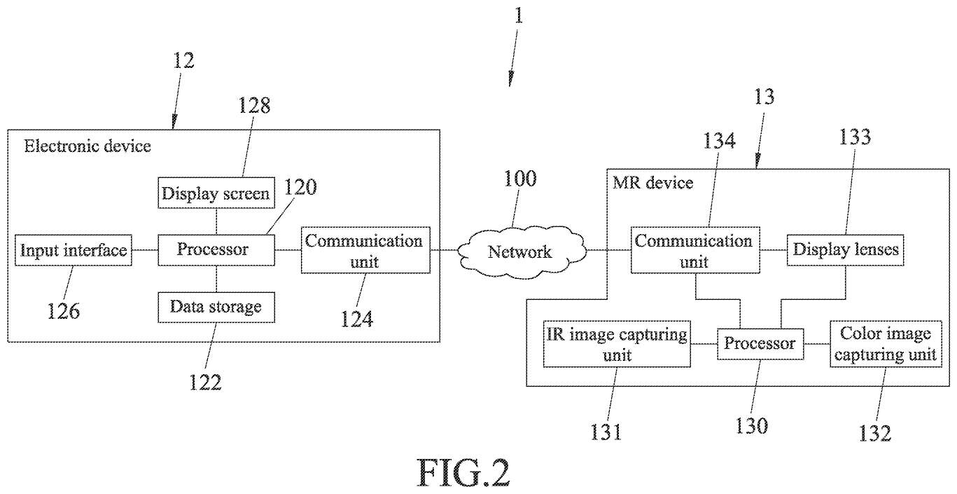

FIG. 2 is a block diagram illustrating components of the system 1 according to one embodiment of the disclosure. The electronic device 12 includes a processor 120, a data storage 122, a communication unit 124, an input interface 126 and a display screen 128.

The processor 120 may include, but not limited to, a single core processor, a multi-core processor, a dual-core mobile processor, a microprocessor, a microcontroller, a digital signal processor (DSP), a field-programmable gate array (FPGA), an application specific integrated circuit (ASIC), and/or a radio-frequency integrated circuit (RFIC), etc. The processor 120 is electrically connected to the data storage 122, the communication unit 124, the input interface 126 and the display screen 128, and is capable of controlling operations of the data storage 122, the communication unit 124, the input interface 126 and the display screen 128.

The data storage 122 may be embodied using one or more of a hard disk, a solid-state drive (SSD) and other non-transitory storage media. The data storage 122 stores a software application including instructions that, when executed by the processor 120, cause the processor 120 to perform operations as described below.

The communication unit 124 may include a short-range wireless communication module supporting a short-range wireless communication network using a wireless technology of Bluetooth® and/or Wi-Fi, etc., and a mobile communication module supporting telecommunication using Long-Term Evolution (LTE), the third generation (3G), fourth generation (4G) and/or fifth generation (5G) of wireless mobile telecommunications technology, and/or the like.

The input interface 126 may be embodied using a physical keyboard, a virtual keyboard, and/or a microphone, etc. In some embodiments, the input interface 126 and the display screen 128 are integrated in the form of a touchscreen.

The MR device 13 may be embodied using a wearable device that is to be worn by a user (e.g., a surgeon performing a surgical procedure), and includes a processor 130, an infrared (IR) image capturing unit 131, a color image capturing unit 132, a display lens 133 and a communication unit 134.

The processor 130 may include, but not limited to, a single core processor, a multi-core processor, a dual-core mobile processor, a microprocessor, a microcontroller, a digital signal processor (DSP), a field-programmable gate array (FPGA), an application specific integrated circuit (ASIC), and/or a radio-frequency integrated circuit (RFIC), etc. The processor 130 is electrically connected to the IR image capturing unit 131, the color image capturing unit 132, the display lens 133, and the communication unit 134, and is capable of controlling operations of the IR image capturing unit 131, the color image capturing unit 132, the display lens 133, and the communication unit 134.

The IR image capturing unit 131 may be embodied using a thermographic camera. In this embodiment, two or more thermographic cameras may be present. The color image capturing unit 132 may be embodied using a digital camera. The display lens 133 may be a transparent lens that enables the user wearing the MR device 13 to see through, and is configured to be controlled by the processor 130 to display an image thereon such that to the perception of the user, the image is superimposed onto real life objects seen through the display lens 133 in the physical world to form a mixed reality view perceived by the user.

The communication unit 134 may include a short-range wireless communication module supporting a short-range wireless communication network using a wireless technology of Bluetooth® and/or Wi-Fi, etc., and a mobile communication module supporting telecommunication using Long-Term Evolution (LTE), the third generation (3G) and/or fourth generation (4G) of wireless mobile telecommunications technology, and/or the like.

The communication unit 124 of the electronic device 12 and the communication unit 134 of the MR device 13 are configured to use the same wireless technology to communicate with each other, and may be controlled to communicate with each other via a network 100 (e.g., the Internet) or via a wireless connection such as Wi-Fi, Bluetooth®, near-field communication (NFC), etc. In some embodiments, the electronic device 12 and the MR device 13 may be electrically connected via a wired connection.

It is noted that in some embodiments, the MR device 13 may include two IR image capturing units 131, two color image capturing units 132, and two display lenses 133 for the two eyes of the user.

In use, the system 1 may be utilized in a surgical procedure performed on a subject (e.g., a human patient).

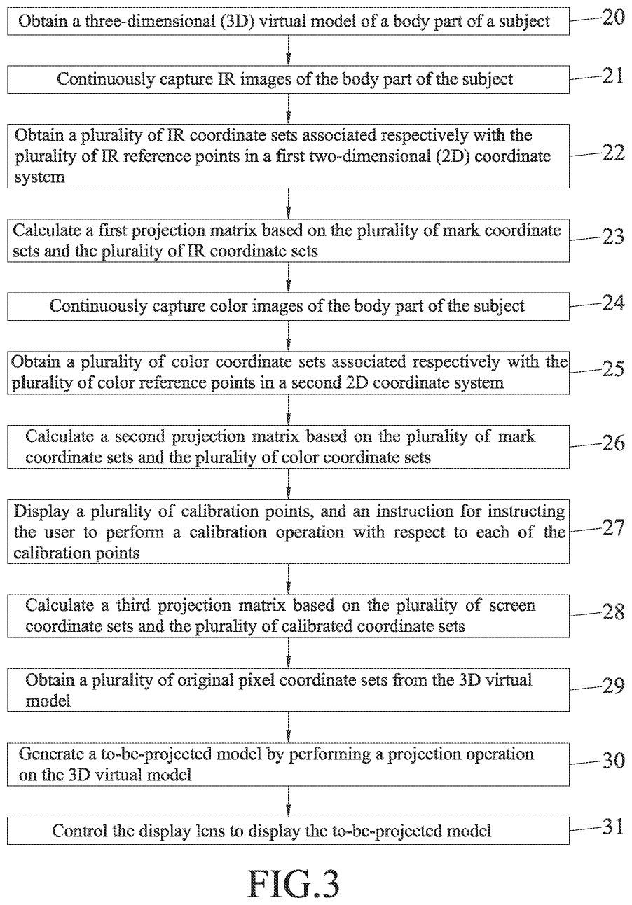

FIG. 3 is a flowchart illustrating steps of a method for performing surgical imaging based on mixed reality according to one embodiment of the disclosure. In this embodiment, the method is implemented using the system 1 as shown in FIGS. 1 and 2.

Prior to the actual surgical procedure, in step 20, the processor 120 obtains a three-dimensional (3D) virtual model of a body part of the subject. The 3D virtual model includes a plurality of model reference points that are associated with a plurality of marks on the body part, respectively. The body part is a part that requires surgery.

In this embodiment, the 3D virtual model is generated or reconstructed using surgical imaging procedures such as magnetic resonance imaging (MRI), computed tomography (CT), digital imaging and communications in medicine (DICOM), etc. The model reference points may be manually designated by the user on the 3D virtual model, and the marks may be manually and physically placed or made on the body part of the subject to correspond respectively with the model reference points in location. For example, in this embodiment, twelve model reference points are designated on an outer surface of the 3D virtual model, and twelve marks are placed on the skin of the body part of the subject. An image of the body part of the subject with the marks is captured and stored in the data storage 122, along with the 3D virtual model.

Afterward, the processor 120 may calculate a plurality of mark coordinate sets that are associated respectively with the plurality of marks on the body part of the subject in a global 3D coordinate system, and a plurality of reference coordinate sets that are associated respectively with the plurality of model reference points in a local 3D coordinate system. In the global 3D coordinate system, the origin may be designated as one of the marks, and in the local 3D coordinate system, the origin may be designated as one of the model reference points.

It is noted that in some embodiments, the 3D virtual model and the image of the body part of the subject with the marks may be generated by an external device and transmitted to the electronic device 12. Additionally, the plurality of mark coordinate sets and the plurality of reference coordinate sets may be calculated by the external device and transmitted to the electronic device 12.

In step 21, the processor 130 of the MR device 13 controls the IR image capturing unit 131 to continuously capture IR images of the body part of the subject. Each of the IR images of the body part includes a plurality of IR reference points that correspond in location with the marks on the body part, respectively.

In step 22, the processor 130 of the MR device 13 obtains a plurality of IR coordinate sets associated respectively with the plurality of IR reference points in a first two-dimensional (2D) coordinate system (since the IR images are two dimensional). In this embodiment, the plurality of IR coordinate sets may be calculated using a library program such as OOOPDS written in C/C++.

Afterward, the processor 130 controls the communication unit 134 to transmit the plurality of IR coordinate sets to the electronic device 12.

In step 23, the processor 120 of the electronic device 12 calculates a first projection matrix P based on the plurality of mark coordinate sets and the plurality of IR coordinate sets.

Specifically, the first projection matrix P₁ may be expressed using the following equation:

P₁=K₁·[R₁|T₁]

where K₁ represents a first intrinsic parameter matrix, and [R₁|T₁] represents a first extrinsic parameter matrix. The first intrinsic parameter matrix K₁ and the first extrinsic parameter matrix [R₁|T₁] may be expressed using the following equations:

$${K}_{1} = [ \begin{matrix}{f}_{x} & s & {c}_{x} \\ 0 & {f}_{y} & {c}_{y} \\ 0 & 0 & 1\end{matrix} ] , [ {R}_{1} \vert {T}_{1} ] = [ \begin{matrix}{v}_{11} & {v}_{12} & {v}_{13} & {w}_{x} \\ {v}_{21} & {v}_{22} & {v}_{23} & {w}_{y} \\ {v}_{31} & {v}_{32} & {v}_{33} & {w}_{z}\end{matrix} ] .$$

It is noted that the effect of the first projection matrix P₁ is to project the mark coordinate set of one of the marks in the global 3D coordinate system to the IR coordinate set of the corresponding one of the IR reference points in the first 2D coordinate system. As such, a relation among the mark coordinate set, the first projection matrix P₁ and the corresponding IR coordinate set may be expressed using the following equation:

$$[ \begin{matrix}{x}_{2} \\ {y}_{2} \\ 1\end{matrix} ] = {P}_{1} [ \begin{matrix}{x}_{1} \\ {y}_{1} \\ {z}_{1} \\ 1\end{matrix} ]$$

where x₁, y₁ and z₁ represent the coordinate values of the mark coordinate set, and x₂ and y₂ represent the coordinate values of the IR coordinate set. That is to say, a matrix of one of the plurality of IR coordinate sets is calculated by performing matrix multiplication of the first projection matrix and a matrix of a corresponding one of the plurality of mark coordinate sets.

In this embodiment, the components of the first intrinsic parameter matrix K₁ and the first extrinsic parameter matrix [R₁|T₁] may be calculated by listing a plurality of linear equations each composed by a respective one of the plurality of IR coordinate sets, the first projection matrix P₁ and the corresponding one of the plurality of mark coordinate sets. Specifically, since in this embodiment, twelve model reference points are designated and twelve marks are placed on the body part of the subject, and each pair of an IR reference point and a mark may yield one linear equation, a linear equation system of twelve equations may be constructed. Then, the processor 120 solves the linear equation system to obtain the values of the components of the first intrinsic parameter matrix K₁ and the first extrinsic parameter matrix [R₁|T₁], thereby calculating the first projection matrix P₁.

In step 24, the processor 130 of the MR device 13 controls the color image capturing unit 132 to continuously capture color images of the body part of the subject. Each of the color images of the body part includes a plurality of color reference points that correspond in location with the marks on the body part, respectively.

In step 25, the processor 130 of the MR device 13 obtains a plurality of color coordinate sets associated respectively with the plurality of color reference points in a second 2D coordinate system (since the color images are two dimensional). In some embodiments, the second 2D coordinate system is established in a calibrating operation on the color image capturing unit 132 to have a specific association with the first 2D coordinate system (i.e., points of one of the first or second 2D coordinate system may be projected onto the other one of the first or second 2D coordinate system).

In this embodiment, the plurality of color coordinate sets are obtained using the IR images. Specifically, the processor 130 obtains the plurality of color coordinate sets from the color images with reference to the IR reference points in the IR images, in a manner similar of that as described in step 22. In other embodiments, the processor 130 may perform an image processing operation on the color images to obtain the plurality of color coordinate sets based on the association between the first and second 2D coordinate systems.

Afterward, the processor 130 controls the communication unit 134 to transmit the plurality of color coordinate sets to the electronic device 12.

In step 26, the processor 120 of the electronic device 12 calculates a second projection matrix P₂ based on the plurality of mark coordinate sets and the plurality of color coordinate sets.

Specifically, the second projection matrix P₂ may be expressed using the following equation:

P₂=K₂·[R₂|T₂]

where K₂ represents a second intrinsic parameter matrix, and [R₂|T₂] represents a second extrinsic parameter matrix. The second intrinsic parameter matrix K₂ and the second extrinsic parameter matrix [R₂|T₂] may be expressed using the following equations:

$${K}_{2} = [ \begin{matrix}{f}_{x}^{′} & {s}^{′} & {c}_{x}^{′} \\ 0 & {f}_{y}^{′} & {c}_{y}^{′} \\ 0 & 0 & 1\end{matrix} ] , [ {R}_{2} \vert {T}_{2} ] = [ \begin{matrix}{v}_{11}^{′} & {v}_{12}^{′} & {v}_{13}^{′} & {w}_{x}^{′} \\ {v}_{21}^{′} & {v}_{22}^{′} & {v}_{23}^{′} & {w}_{y}^{′} \\ {v}_{31}^{′} & {v}_{32}^{′} & {v}_{33}^{′} & {w}_{z}^{′}\end{matrix} ] .$$

It is noted that the effect of the second projection matrix P₂ is to project the mark coordinate set of one of the marks in the global 3D coordinate system to the color coordinate set of the corresponding one of the color reference points in the second 2D coordinate system. It should be noted that the color reference points in the second 2D coordinate system may be projected respectively as points in the first 2D coordinate system. As such, a relation among the mark coordinate set, the second projection matrix P₂ and the corresponding color coordinate set may be expressed using the following equation:

$$[ \begin{matrix}{x}_{3} \\ {y}_{3} \\ 1\end{matrix} ] = {P}_{2} [ \begin{matrix}{x}_{1} \\ {y}_{1} \\ {z}_{1} \\ 1\end{matrix} ]$$

where x₃ and y₃ represent the coordinate values of the color coordinate set. That is to say, a matrix of one of the plurality of color coordinate sets is calculated by performing matrix multiplication of the second projection matrix P₂ and a matrix of a corresponding one of the plurality of mark coordinate sets.

In this embodiment, the components of the second intrinsic parameter matrix K₂ and the second extrinsic parameter matrix [R₂|T₂] may be calculated by listing a plurality of linear equations each composed by a respective one of the plurality of color coordinate sets, the second projection matrix P₂ and the corresponding one of the plurality of mark coordinate sets. Specifically, since in this embodiment, twelve model reference points are designated and twelve marks are placed on the body part of the subject, and each pair of a color reference point and a mark may yield one linear equation, a linear equation system of twelve equations may be constructed. Then, the processor 120 solves the linear equation system to obtain the values of the components of the second intrinsic parameter matrix K₂ and the second extrinsic parameter matrix [R₂|T₂], thereby calculating the second projection matrix P₂.

In step 27, the processor 130 of the MR device 13 controls the display lens 133 to display a plurality of calibration points, and an instruction (in the form of, for example, a visible message, etc.) for instructing the user to perform a calibration operation with respect to each of the calibration points. In this embodiment, twelve calibration points are displayed.

Specifically, the user may be instructed to manually hold a calibration object (e.g., a physical black dot), and to move the calibration object such that to the perception of the user, the calibration object overlaps with one of the calibration points, thereby completing the calibration operation with respect to said one of the calibration points. Afterward, the user is instructed to move the calibration object with respect to each of the other calibration points displayed on the display lens 133 to complete the calibration operation with respect to all of the calibration points.

In response, the processor 130 obtains a plurality of screen coordinate sets associated respectively with the plurality of calibration points in a third 2D coordinate system with respect to the display lens 133, and a plurality of calibrated coordinate sets associated with the calibration points in the global 3D coordinate system. It is noted that each of the calibrated coordinate sets may be a coordinate set of the calibration object (overlapping one of the calibration points) in the global 3D coordinate system.

In another embodiment where the display lens 133 is embodied as a transparent touchscreen, the calibration operation may be done by the user placing the calibration object in an arbitrary location that can be perceived by the user through the display lens 133, and subsequently inputting a corresponding location on the display lens 133 that overlaps with the calibration object (by, for example, directly clicking or tapping on the corresponding location) to serve as the calibration point. Such operation may be repeated multiple times.

As such, the processor 130 obtains the plurality of calibrated coordinate sets associated with the arbitrary locations of the calibration object in the global 3D coordinate system, and obtains the plurality of screen coordinate sets associated respectively with the corresponding locations on the display lens 133 inputted by the user.

Afterward, the processor 130 controls the communication unit 134 to transmit the plurality of calibrated coordinate sets and the plurality of screen coordinate sets to the electronic device 12.

In step 28, the processor 120 of the electronic device 12 calculates a third projection matrix P₃ based on the plurality of screen coordinate sets and the plurality of calibrated coordinate sets.

Specifically, the third projection matrix P₃ may be expressed using the following equation:

P₃=K₃·[R₃|T₃]

where K₃ represents a third intrinsic parameter matrix, and [R₃|T₃] represents a third extrinsic parameter matrix. The third intrinsic parameter matrix K₃ and the third extrinsic parameter matrix [R₃|T₃] may be expressed using the following equations:

$${K}_{3} = [ \begin{matrix}{f}_{x}^{″} & {s}^{″} & {c}_{x}^{″} \\ 0 & {f}_{y}^{″} & {c}_{y}^{″} \\ 0 & 0 & 1\end{matrix} ] , [ {R}_{3} \vert {T}_{3} ] = [ \begin{matrix}{v}_{11}^{″} & {v}_{12}^{″} & {v}_{13}^{″} & {w}_{x}^{″} \\ {v}_{21}^{″} & {v}_{22}^{″} & {v}_{23}^{″} & {w}_{y}^{″} \\ {v}_{31}^{″} & {v}_{32}^{″} & {v}_{33}^{″} & {w}_{z}^{″}\end{matrix} ] .$$

It is noted that the effect of the third projection matrix P₁ is to project the mark coordinate set of one of the marks in the global 3D coordinate system to the screen coordinate set of the corresponding one of the calibration points on the display lens 133. As such, a relation among the mark coordinate set, the second projection matrix P₃ and the corresponding screen coordinate set may be expressed using the following equation:

$$[ \begin{matrix}{x}_{4} \\ {y}_{4} \\ 1\end{matrix} ] = {P}_{3} [ \begin{matrix}{x}_{1} \\ {y}_{1} \\ {z}_{1} \\ 1\end{matrix} ]$$

where x₄ and y₄ represent the coordinate values of the screen coordinate set. That is to say, a matrix of one of the plurality of screen coordinate sets is calculated by performing matrix multiplication of the third projection matrix P₃ and a matrix of a corresponding one of the plurality of mark coordinate sets.

In this embodiment, the components of the third intrinsic parameter matrix K₃ and the third extrinsic parameter matrix [R₃|T₃] may be calculated by listing a plurality of linear equations each composed by a respective one of the plurality of color coordinate sets, the third projection matrix P₃ and the corresponding one of the plurality of mark coordinate sets. Specifically, since in this embodiment, 12 calibration points are designated, and each pair of a calibration point and a mark may yield one linear equation, a linear equation system of 12 equations may be constructed. Then, the processor 120 solves the linear equation system to obtain the values of the components of the third intrinsic parameter matrix K₃ and the third extrinsic parameter matrix [R₃|T₃], therefore calculating the third projection matrix P₃.

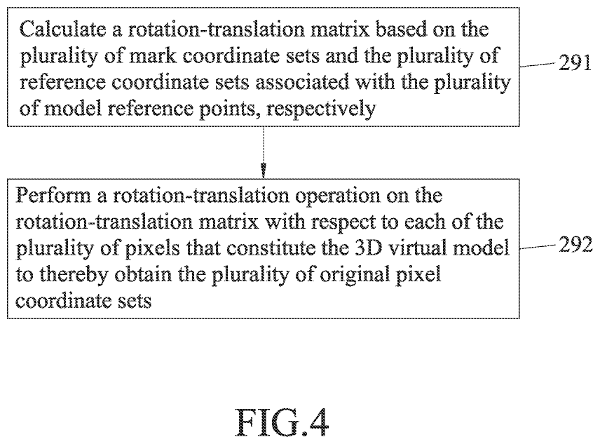

In step 29, the processor 120 of the electronic device 12 obtains a plurality of original pixel coordinate sets from the 3D virtual model. The plurality of original pixel coordinate sets are associated respectively with a plurality pixels that constitute the 3D virtual model in the global 3D coordinate system. Specifically, the operation of step 29 may be illustrated in a flowchart of FIG. 4.

In sub-step 291, the processor 120 of the electronic device 12 calculates a rotation-translation matrix [R₄|T₄] based on the plurality of mark coordinate sets and the plurality of reference coordinate sets associated with the plurality of model reference points, respectively. The rotation-translation matrix [R₄|T₄] may be expressed using the following equation:

$$[ {R}_{4} \vert {T}_{4} ] = [ \begin{matrix}{v}_{11}^{′′′} & {v}_{12}^{′′′} & {v}_{13}^{′′′} & {w}_{x}^{′′′} \\ {v}_{21}^{′′′} & {v}_{22}^{′′′} & {v}_{23}^{′′′} & {w}_{y}^{′′′} \\ {v}_{31}^{′′′} & {v}_{32}^{′′′} & {v}_{33}^{′′′} & {w}_{z}^{′′′} \\ 0 & 0 & 0 & 1\end{matrix} ] .$$

Specifically, the rotation-translation matrix [R₄|T₄] represents a shift from one of the mark coordinate sets to a corresponding one of the reference coordinate sets. As such, a relation among the mark coordinate set, the rotation-translation matrix [R₄|T₄] and the corresponding reference coordinate set may be expressed using the following equation:

$$[ \begin{matrix}{x}_{5} \\ {y}_{5} \\ {z}_{5} \\ 1\end{matrix} ] = [ {R}_{4} \vert {T}_{4} ] [ \begin{matrix}{x}_{1} \\ {y}_{1} \\ {z}_{1} \\ 1\end{matrix} ] ,$$

where x₅, y₅ and z₅ represent the coordinate values of the screen coordinate set. That is to say, a matrix of one of the plurality of screen coordinate sets is calculated by performing matrix multiplication of the rotation-translation matrix [R₄|T₄] and a matrix of a corresponding one of the plurality of mark coordinate sets.

In this embodiment, the components of the rotation-translation matrix [R₄|T₄] may be calculated by listing a plurality of linear equations each composed by a respective one of the plurality of screen coordinate sets, the rotation-translation matrix [R₄|T₄] and the corresponding one of the plurality of mark coordinate sets. Specifically, since in this embodiment, twelve calibration points are designated, and each pair of calibration point and mark may yield one linear equation, a linear equation system of twelve equations may be constructed. Then, the processor 120 solves the linear equation system to obtain the values of the components of the rotation-translation matrix [R₄|T₄].

In sub-step 292, the processor 120 performs a rotation-translation operation on the rotation-translation matrix [R₄|T₄] with respect to each of the plurality of pixels that constitute the 3D virtual model to thereby obtain the plurality of original pixel coordinate sets. It is noted that, for each of the plurality of pixels, a specific coordinate set in the global 3D coordinate system may be obtained using the library program such as OOOPDS written in C/C++.

Afterward, in step 30, the processor 120 of the electronic device 12 generates a to-be-projected model by performing a projection operation on the 3D virtual model. The projection operation is performed based on the plurality of original pixel coordinate sets each associated with one of the pixels of the 3D virtual model in the global 3D coordinate system, the first projection matrix P₁, the second projection matrix P₂, and the third projection matrix P₃.

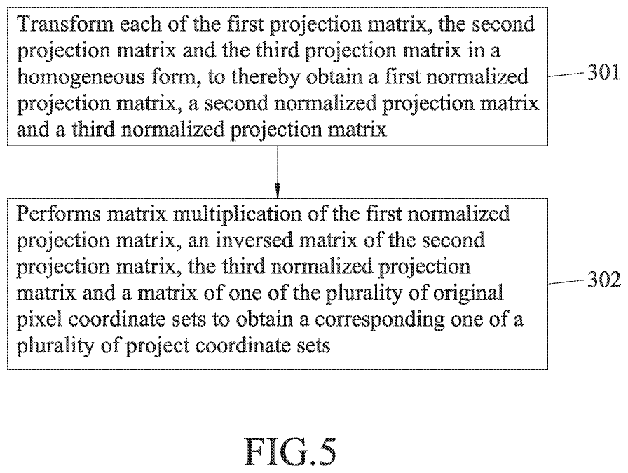

Specifically, the operation of step 30 may be illustrated in a flowchart of FIG. 5.

In sub-step 301, the processor 120 transforms each of the first projection matrix P₁, the second projection matrix P₂ and the third projection matrix P₃ in a homogeneous form, to thereby obtain a first normalized projection matrix H₁, a second normalized projection matrix H₂ and a third normalized projection matrix H₃.

In this embodiment, the first, second and third normalized projection matrices may be calculated using the following equations, respectively:

$${H}_{1} = [ \frac{{P}_{1}}{0001} ]$$

$${H}_{2} = [ \frac{{P}_{2}}{0001} ]$$

Afterward, in sub-step 302, the processor 120 performs matrix multiplication of the first normalized projection matrix H₁, an inversed matrix of the second projection matrix H₂⁻¹, the third normalized projection matrix H₃ and a matrix of one of the plurality of original pixel coordinate sets to obtain a corresponding one of a plurality of project coordinate sets. The plurality of project coordinate sets are associated respectively with a plurality of pixels that constitute the to-be-projected model.

Specifically, a relation among a project coordinate set, the normalized projection matrices and the corresponding original coordinate set may be expressed using the following equation:

$$[ \begin{matrix}u \\ v \\ 1\end{matrix} ] = {H}_{1} \cdot {H}_{2}^{- 1} \cdot {H}_{3} [ \begin{matrix}{x}_{0} \\ {y}_{0} \\ {z}_{0}\end{matrix} ]$$

where u and v represent the coordinate values of the project coordinate set, and x₀, y₀ and z₀ represent the coordinate values of the original coordinate set.

Afterward, the processor 120 controls the communication unit 124 to transmit the to-be-projected model to the MR device 13.

It is noted that in various embodiments, the operations of step 29 may be implemented at an arbitrary time instance prior to step 30, and is not necessarily implemented after step 28.

In step 31, the processor 130 of the MR device 13 controls the display lens 133 to display the to-be-projected model. In this manner, the to-be-projected model is accurately projected onto the body part of the subject to form a mixed reality view perceived by the user, enabling the user to perform the surgical procedure with improved convenience.

According to one embodiment, the system 1 may include only the MR device 13. The MR device 13 may include a data storage that stores the software application as described above. When executed by the processor 130, the software application causes the processor 130 to perform the steps of the method as described in FIG. 3.

According to one embodiment of the disclosure, there is provided a non-transitory computer-readable storage medium storing instructions that, when executed by a processor of an electronic device communicating with an MR device, cause the processor to perform steps of a method as described in FIG. 3.

To sum up, the embodiments of the disclosure provide a method and a system 1 for performing surgical imaging based on mixed reality. By using the IR images of the body part, the color images of the body part, and the calibration operation, the system 1 is able to determine a series of shifts for projecting the 3D virtual model accurately onto the body part of the subject, from the perception of the user looking through the display lens 133. In this manner, the need for the user to look away from the body part of the subject while performing the surgical procedure is eliminated, and therefore the surgical procedure can be performed with improved convenience and accuracy.

In the description above, for the purposes of explanation, numerous specific details have been set forth in order to provide a thorough understanding of the embodiments. It will be apparent, however, to one skilled in the art, that one or more other embodiments may be practiced without some of these specific details. It should also be appreciated that reference throughout this specification to “one embodiment,” “an embodiment,” an embodiment with an indication of an ordinal number and so forth means that a particular feature, structure, or characteristic may be included in the practice of the disclosure. It should be further appreciated that in the description, various features are sometimes grouped together in a single embodiment, figure, or description thereof for the purpose of streamlining the disclosure and aiding in the understanding of various inventive aspects, and that one or more features or specific details from one embodiment may be practiced together with one or more features or specific details from another embodiment, where appropriate, in the practice of the disclosure.

While the disclosure has been described in connection with what is are considered the exemplary embodiments, it is understood that this disclosure is not limited to the disclosed embodiments but is intended to cover various arrangements included within the spirit and scope of the broadest interpretation so as to encompass all such modifications and equivalent arrangements.

What is claimed is:

1. A method for performing surgical imaging based on mixed reality (MR) to be implemented using a system that includes an MR device to be worn by a user, the MR device including a processor, an infrared (IR) image capturing unit, a color image capturing unit and a display lens, the method comprising:
a) obtaining, by the processor, a three-dimensional (3D) virtual model of a body part of a subject, the 3D virtual model including a plurality of model reference points that are associated with a plurality of marks on the body part, respectively;
b) controlling, by the processor, the IR image capturing unit to continuously capture IR images of the body part of the subject, each of the IR images of the body part including a plurality of IR reference points that correspond in location with the marks on the body part, respectively;
c) calculating, by the processor, a first projection matrix based on a plurality of mark coordinate sets associated respectively with the plurality of marks on the body part in a global 3D coordinate system, and a plurality of IR coordinate sets associated respectively with the plurality of IR reference points in a first two-dimensional (2D) coordinate system;
d) controlling, by the processor, the color image capturing unit to continuously capture color images of the body part of the subject, each of the color images of the body part including a plurality of color reference points that correspond in location with the marks on the body part, respectively;
e) obtaining, by the processor, a plurality of color coordinate sets associated respectively with the plurality of color reference points in a second 2D coordinate system;
f) calculating, by the processor, a second projection matrix based on the plurality of mark coordinate sets and the plurality of color coordinate sets;
g) controlling, by the processor, the display lens to display a plurality of calibration points, and an instruction for instructing the user to perform a calibration operation with respect to each of the calibration points, to thereby obtain a plurality of screen coordinate sets that are associated respectively with the plurality of calibration points on the display lens and a plurality of calibrated coordinate sets that are associated with the calibration points in the global 3D coordinate system;
h) calculating, by the processor, a third projection matrix based on the plurality of screen coordinate sets and the plurality of calibrated coordinate sets;
i) generating, by the processor, a to-be-projected model by performing a projection operation on the 3D virtual model, the projection operation being performed based on a plurality of original pixel coordinate sets, the first projection matrix, the second projection matrix and the third projection matrix, the plurality of original pixel coordinate sets being associated respectively with a plurality of pixels that constitute the 3D virtual model in the global 3D coordinate system; and
j) controlling, by the processor, the display lens to display the to-be-projected model.

2. The method of claim 1, wherein step c) includes calculating, for each of the plurality of IR coordinate sets, a matrix of the IR coordinate set by performing matrix multiplication of the first projection matrix and a matrix of a corresponding one of the plurality of mark coordinate sets, and calculating the first projection matrix based on the plurality of mark coordinate sets and the matrices of the plurality of IR coordinate sets.

3. The method of claim 1, wherein step e) includes obtaining the plurality of color coordinate sets from the color images with reference to the IR reference points in the IR images.

4. The method of claim 1, wherein step f) includes calculating, for each of the plurality of color coordinate sets, a matrix of the color coordinate set by performing matrix multiplication of the second projection matrix and a matrix of a corresponding one of the plurality of mark coordinate sets, and calculating the second projection matrix based on the plurality of mark coordinate sets and the matrices of the plurality of color coordinate sets.

5. The method of claim 1, wherein step h) includes calculating, for each of the plurality of screen coordinate sets, a matrix of the screen coordinate set by performing matrix multiplication of the third projection matrix and a corresponding one of the plurality of mark coordinate sets, and calculating the third projection matrix based on the plurality of screen coordinate sets and the matrices of the plurality of screen coordinate sets.

6. The method of claim 1, further comprising, prior to step i):
calculating, by the processor, a rotation-translation matrix based on the plurality of mark coordinate sets and a plurality of reference coordinate sets that are associated with the plurality of model reference points, respectively; and
performing a rotation-translation operation using the rotation-translation matrix with respect to each of the plurality of pixels that constitute the 3D virtual model, to thereby obtain the plurality of original pixel coordinate sets.

7. The method of claim 1, wherein step i) includes:
transforming each of the first projection matrix, the second projection matrix and the third projection matrix in a homogeneous form to thereby obtain a first normalized projection matrix, a second normalized projection matrix and a third normalized projection matrix; and
performing matrix multiplication of the first normalized projection matrix, an inversed matrix of the second projection matrix, the third normalized projection matrix and a matrix of the plurality of original pixel coordinate sets to obtain a plurality of project coordinate sets associated respectively with a plurality of pixels that constitute the to-be-projected model.

8. A mixed reality (MR) system for performing surgical imaging, comprising an electronic device and an MR device that communicates with said electronic device and that is to be worn by a user, said electronic device including a processor, a data storage, a communication unit, an input interface and a display screen, said MR device including a processor, an infrared (IR) image capturing unit, a color image capturing unit and a display lens, wherein:
said processor of said electronic device is programmed to obtain a three-dimensional (3D) virtual model of a body part of a subject, the 3D virtual model including a plurality of model reference points that are associated with a plurality of marks on the body part, respectively;
said processor of said MR device is programmed to control said IR image capturing unit to continuously capture IR images of the body part of the subject, each of the IR images of the body part including a plurality of IR reference points that correspond in location with the marks on the body part, respectively;
said processor of said electronic device is programmed to obtain a first projection matrix based on a plurality of mark coordinate sets associated respectively with the plurality of marks on the body part in a global 3D coordinate system, and a plurality of IR coordinate sets associated respectively with the plurality of IR reference points in a first two-dimensional (2D) coordinate system;
said processor of said MR device is programmed to control said color image capturing unit to continuously capture color images of the body part of the subject, each of the color images of the body part including a plurality of color reference points that correspond in location with the marks on the body part, respectively;
said processor of said electronic device is programmed to obtain a plurality of color coordinate sets associated respectively with the plurality of color reference points in a second 2D coordinate system, and to calculate a second projection matrix based on the plurality of mark coordinate sets and the plurality of color coordinate sets;
said processor of said MR device is programmed to control said display lens to display a plurality of calibration points, and an instruction for instructing the user to perform a calibration operation with respect to each of the calibration points, to thereby obtain a plurality of screen coordinate sets that are associated respectively with the plurality of calibration points on said display lens and a plurality of calibrated coordinate sets that are associated with the calibration points in the global 3D coordinate system;
said processor of said electronic device is programmed to calculate a third projection matrix based on the plurality of screen coordinate sets and the plurality of calibrated coordinate sets, and to generate a to-be-projected model by performing a projection operation on the 3D virtual model, the projection operation being performed based on a plurality of original pixel coordinate sets, the first projection matrix, the second projection matrix and the third projection matrix, the plurality of original pixel coordinate sets being associated respectively with a plurality of pixels that constitute the 3D virtual model in the global 3D coordinate system; and
said processor of said MR device is programmed to control said display lens to display the to-be-projected model.

9. The MR system of claim 8, wherein said processor of said electronic device is programmed to calculate, for each of the plurality of IR coordinate sets, a matrix of the IR coordinate set by performing matrix multiplication of the first projection matrix and a matrix of a corresponding one of the plurality of mark coordinate sets, and to obtain the first projection matrix based on the plurality of mark coordinate sets and the matrices for the plurality of IR coordinate sets.

10. The MR system of claim 8, wherein said processor of said electronic device is programmed to obtain the plurality of color coordinate sets from the color images with reference to the IR reference points in the IR images.

11. The MR system of claim 8, wherein said processor of said electronic device is programmed to calculate, for each of the plurality of color coordinate sets, a matrix of the color coordinate set by performing matrix multiplication of the second projection matrix and a matrix of a corresponding one of the plurality of mark coordinate sets, and to calculate the second projection matrix based on the plurality of mark coordinate sets and the matrices of the plurality of color coordinate sets.

12. The MR system of claim 8, wherein said processor of said electronic device is programmed to calculate, for each of the plurality of screen coordinate sets, a matrix of the coordinate set by performing matrix multiplication of the third projection matrix and a corresponding one of the plurality of mark coordinate sets, and to calculate the third projection matrix based on the plurality of screen coordinate sets and the matrices of the plurality of calibrated coordinate sets.

13. The MR system of claim 8, wherein said processor of said electronic device is further programmed to:
calculate a rotation-translation matrix based on the plurality of mark coordinate sets and a plurality of reference coordinate sets that are associated with the plurality of model reference points, respectively; and
perform a rotation-translation operation using the rotation-translation matrix with respect to each of the plurality of pixels that constitute the 3D virtual model to thereby obtain the plurality of original pixel coordinate sets.

14. The MR system of claim 8, wherein said processor of said electronic device is programmed to, in generating the to-be-projected model:
transform each of the first projection matrix, the second projection matrix and the third projection matrix in a homogeneous form to thereby obtain a first normalized projection matrix, a second normalized projection matrix and a third normalized projection matrix; and
perform matrix multiplication of the first normalized projection matrix, an inversed matrix of the second projection matrix, the third normalized projection matrix and a matrix of the plurality of original pixel coordinate sets to obtain a plurality of project coordinate sets associated respectively with a plurality of pixels that constitute the to-be-projected model.

15. A non-transitory computer-readable storage medium storing instructions that, when executed by a processor of an electronic device communicating with a mixed reality device, cause the processor to perform steps of the method of claim 1.
